(19) United States
(12) Patent Application Publication
(10) Pub. No.: US 2011/0105845 A1
(43) Pub. Date: May 5, 2011

(54) ENDOSCOPIC DEVICE WITH FLUID CLEANING

(76) Inventors: Tal Gordon, Hod HaSharon (IL); Izhak Fabian, Kfar Truman (IL); David Klein, Rehovot (IL)

(21) Appl. No.: 12/993,285

(22) PCT Filed: May 20, 2009
(86) PCT No.: PCT/US2009/044595

§ 371 Date: Nov. 18, 2010

Related U.S. Application Data
(60) Provisional application No. 61/054,510, filed on May 20, 2008.
(60) Provisional application No. 61/139,647, filed on Dec. 22, 2008.

(51) Int. Cl. A61B 1/12

(52) U.S. Cl. 600/156

(57) ABSTRACT

A method for enhancing performance of an endoscope (212) for use with a body lumen that has not been sufficiently cleaned for viewing with the endoscope (212), including assembling an endoscopic device (100, 400) to an endoscope (212), the endoscopic device (100, 400) including a washing member (201, 404) that provides a flow of cleaning fluid for cleaning a body lumen, and a suction member (206, 406) for draining material from the body lumen, and causing fluid to exit the washing member (201, 404) into the body lumen to clear debris away from a path of the endoscope (212) so that the endoscope (212) can view the exposed body lumen, and draining at least a portion of the debris through the suction member (206, 406).

### FIELD OF THE INVENTION

The present invention relates generally to systems for navigating and imaging body lumens, such as the gastrointestinal (GI) tract, and particularly to a system and method for performing a colonoscopy with self-cleaning features that preclude the need for preparatory cleaning of the colon or assist/clean in cases of poor preparation.

### BACKGROUND OF THE INVENTION

Many imaging devices are known for producing medical images of body lumens, such as the gastrointestinal (GI) tract. For example, endoscopy is widely used for observing, photographing tissue, and taking specimens from lesions and the like. In a conventional method of examining a colon using an endoscope, for example, the endoscope is typically manually inserted into the colon. In this manual technique, patients may often complain of abdominal pain and distention because the colon is extended or excessively dilated, thereby complicating the endoscopic procedure. There may be a risk for the colon to bleed and be accidentally perforated. Insertion of an endoscope through the sigmoid colon and into the descending colon, or through the splenic flexure, the transverse colon, the hepatic flexure or parts affected by previous operations may also be accompanied with difficulty.

### SUMMARY OF THE INVENTION

The present invention seeks to provide an improved endoscopic device for performing endoscopy in a body lumen comprising a cleaning jet lumen (cleaning includes irrigation and/or washing) that provides a jet of cleaning fluid that cleans said body lumen during imaging thereof, such as the gastrointestinal (GI) tract, as is described more in detail hereinbelow. The invention includes a variety of methods/procedures, such as cleaning while imaging, cleaning during insertion of the endoscope, cleaning while withdrawing, and any combinations thereof. The invention is described hereinbelow with reference to the GI tract, but it is understood that the invention is not limited to the GI tract and may be used for other body lumens as well.

There is thus provided in accordance with an embodiment of the present invention, a method for enhancing performance of an endoscope for use with a body lumen that has not been sufficiently cleaned for viewing with the endoscope, including assembling an endoscopic device to an endoscope, the endoscopic device including a washing member that provides a flow of cleaning fluid for cleaning a body lumen, and a suction member for draining material from the body lumen, and causing fluid to exit the washing member into the body lumen to clear debris away from a path of the endoscope so that the endoscope can view the exposed body lumen, and draining at least a portion of the debris through the suction member.

In accordance with an embodiment of the present invention the endoscopic device is assembled as an add-on accessory to the endoscope. Alternatively, the endoscopic device is assembled with the endoscope as a built-in manufactured assembly.

In accordance with an embodiment of the present invention the method further includes clearing an obstruction from at least one of the washing member and the suction member, such as by causing fluid to exit the washing member and/or the suction member to clear the obstruction.

In accordance with an embodiment of the present invention the method further includes causing fluid to exit the washing member to help propel the endoscope in the body lumen.

In accordance with an embodiment of the present invention the method further includes introducing vibratory motion in the endoscope to help propel the endoscope in the body lumen.

In accordance with an embodiment of the present invention the method further includes introducing percussion forces in the endoscope to help propel the endoscope in the body lumen.

In accordance with an embodiment of the present invention the method further includes fitting a discardable sheath over the endoscope.

In accordance with an embodiment of the present invention the method further includes removing the sheath from the endoscope by inverting the sheath backwards over an end of the endoscope.

In accordance with an embodiment of the present invention the method further includes performing a sequence of alternating between spraying jets of fluid through the washing member to clean away debris in the body lumen, sucking the debris away through the suction member, and cleaning debris away from a drain outlet of the suction member.

In accordance with an embodiment of the present invention the method further includes using a control system to automatically control operational parameters of the sequence, the operational parameters including at least one of time durations of each part of the sequence, and frequency and amplitude of spraying the jets of fluid and sucking the debris.

There is thus provided in accordance with an embodiment of the present invention, an endoscopic device for use with an endoscope, including a washing member that provides a flow of cleaning fluid for cleaning a body lumen, a suction member for draining material from the body lumen, and an anti-clogging element that moves with respect to at least one of the washing member and the suction member for clearing an obstruction from at least one of the washing member and the suction member.

In accordance with an embodiment of the present invention the anti-clogging element includes a spray of fluid that exits the washing member or the suction member.

In accordance with an embodiment of the present invention the endoscopic device further includes a control system for operating the endoscopic device, the control system including a controller, one or more pumps and a control valve, wherein the control valve controls suction to the suction member and the one or more pumps pump cleaning fluid to the washing member. In accordance with an embodiment of the present invention a discardable sheath fits over the endoscope.

### BRIEF DESCRIPTION OF THE DRAWINGS

The present invention will be understood and appreciated more fully from the following detailed description taken in conjunction with the drawings in which:

FIGS. 1A-1P are simplified illustrations of an endoscopic device, constructed and operative in accordance with an embodiment of the present invention;

FIG. 2A is a simplified illustration of a vibratory device, constructed and operative in accordance with an embodiment of the present invention, which may be used with any of the endoscopic devices of the present invention to create vibrations and/or percussive forces in the endoscopic device;

FIG. 2B is a simplified illustration of a vibratory device, constructed and operative in accordance with another embodiment of the present invention, which includes a multiplicity of pistons for creating percussive forces along the length of the vibratory device;

FIG. 3A is a simplified illustration of the vibratory device configured to mate with a grinding tool head, in accordance with an embodiment of the present invention;

FIG. 3B is a simplified illustration of the vibratory device configured to mate with a grinding tool head, wherein the distal end of the vibratory device is bent off-center;

FIG. 3C is a simplified illustration of a variation of the grinding tool head formed with channels for cleaning fluid to flow therethrough, in accordance with an embodiment of the present invention; and

FIGS. 4A-4B are simplified illustrations of an endoscopic device, constructed and operative in accordance with another embodiment of the present invention.

### DETAILED DESCRIPTION OF EMBODIMENTS

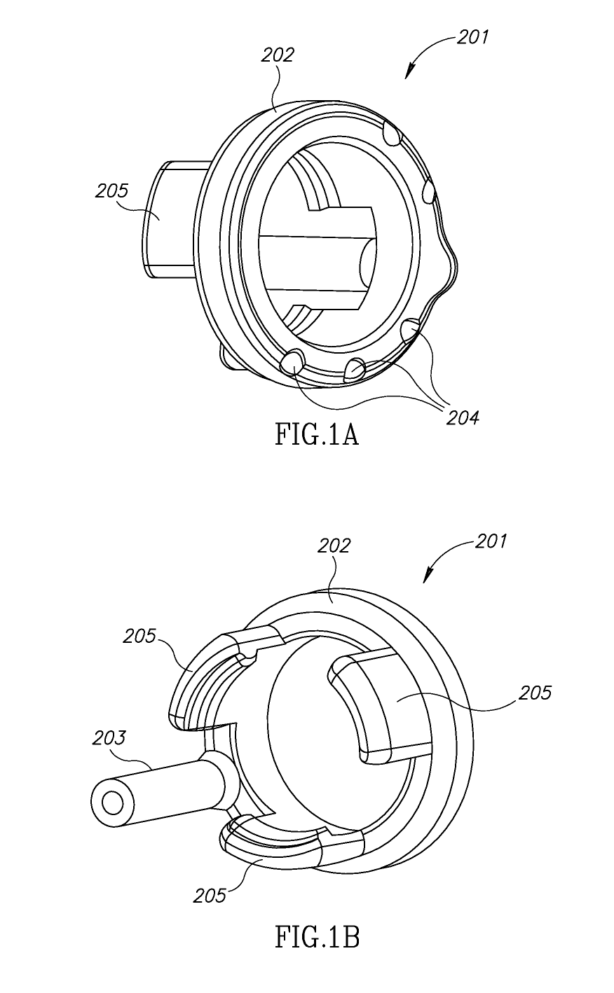

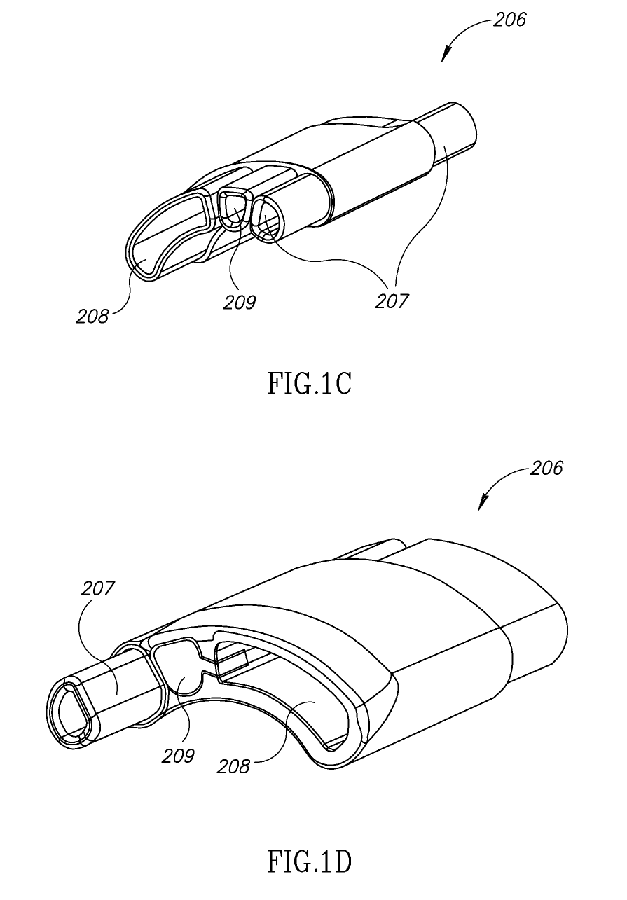

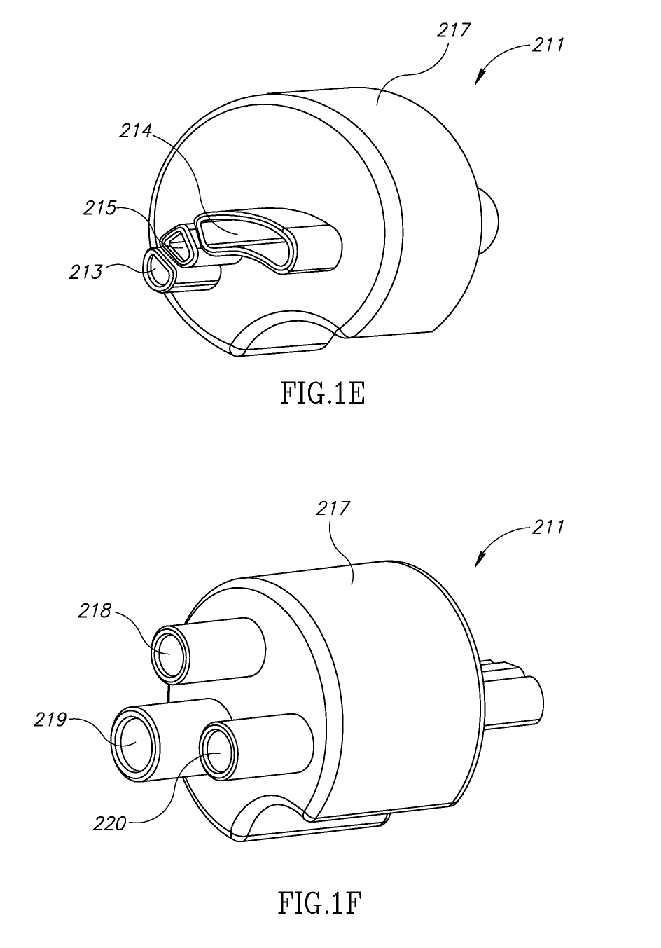

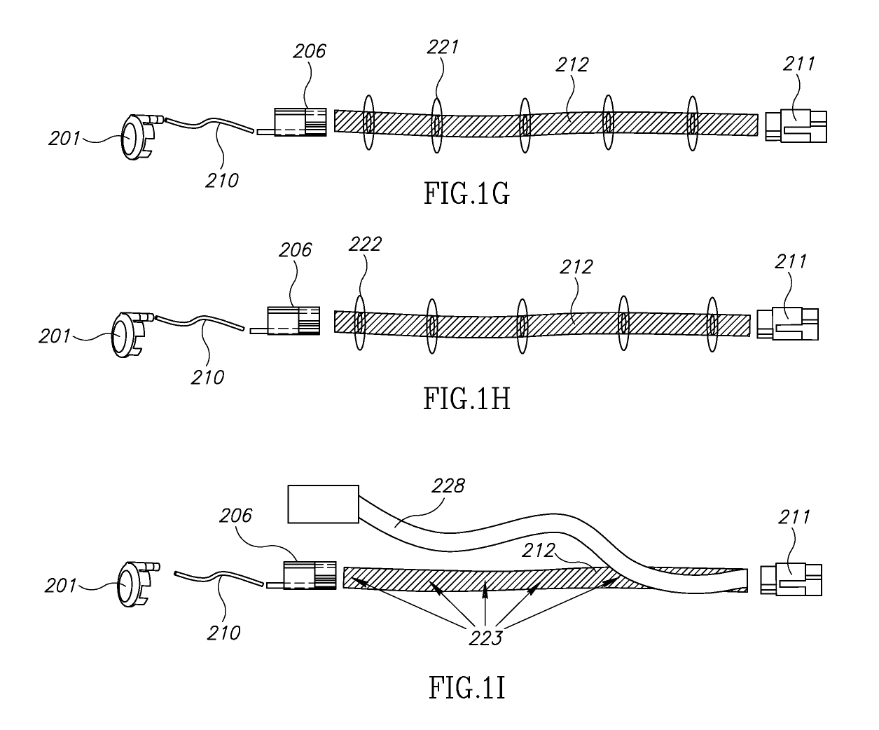

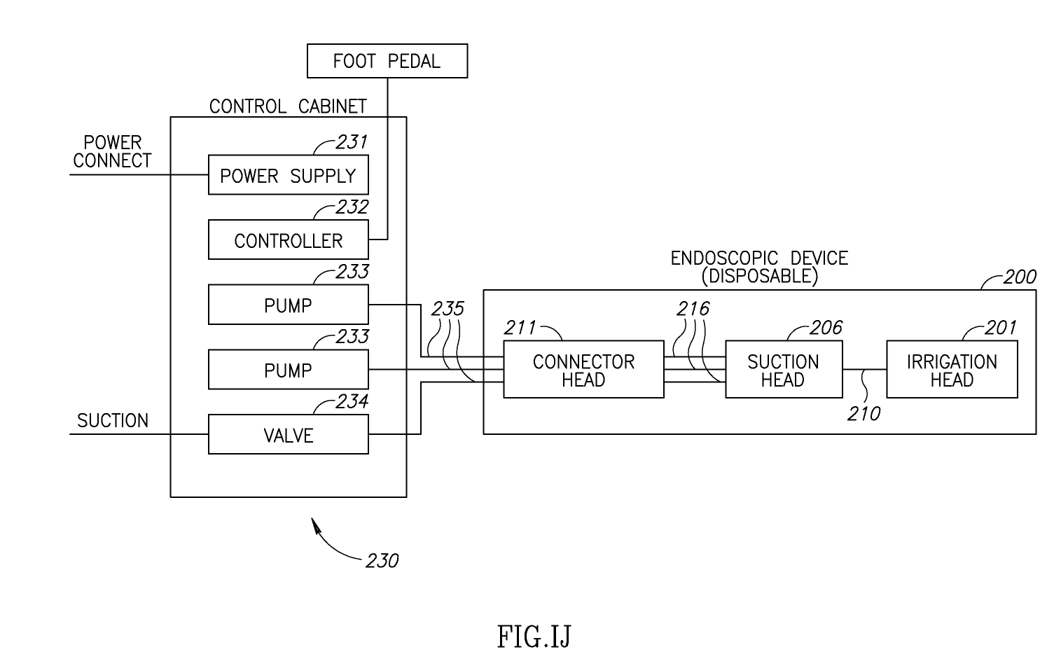

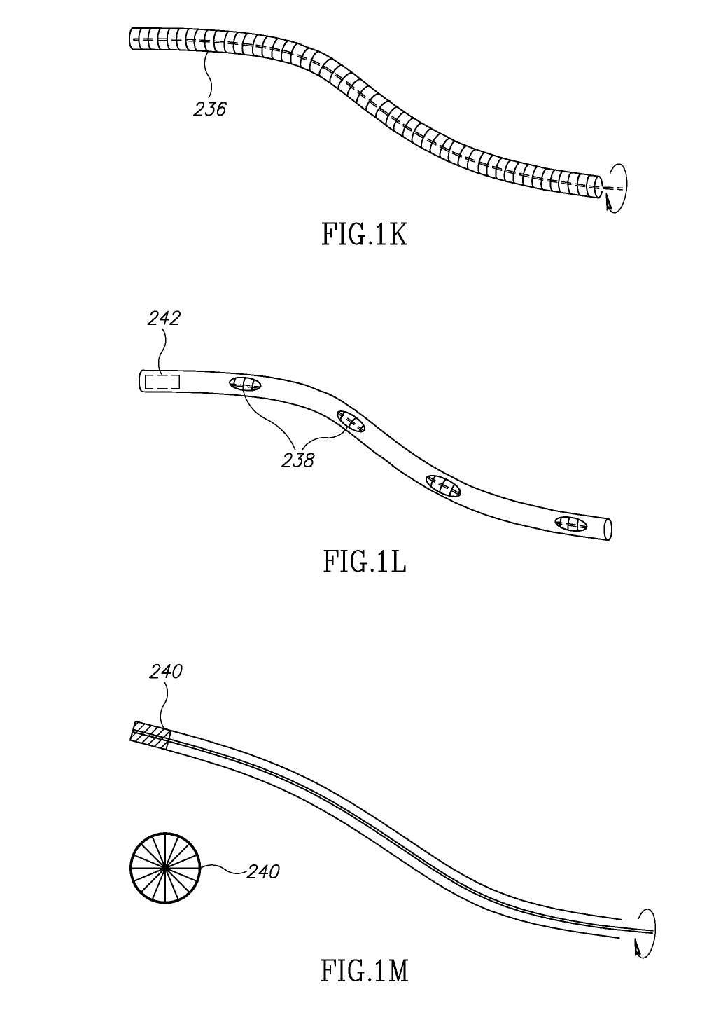

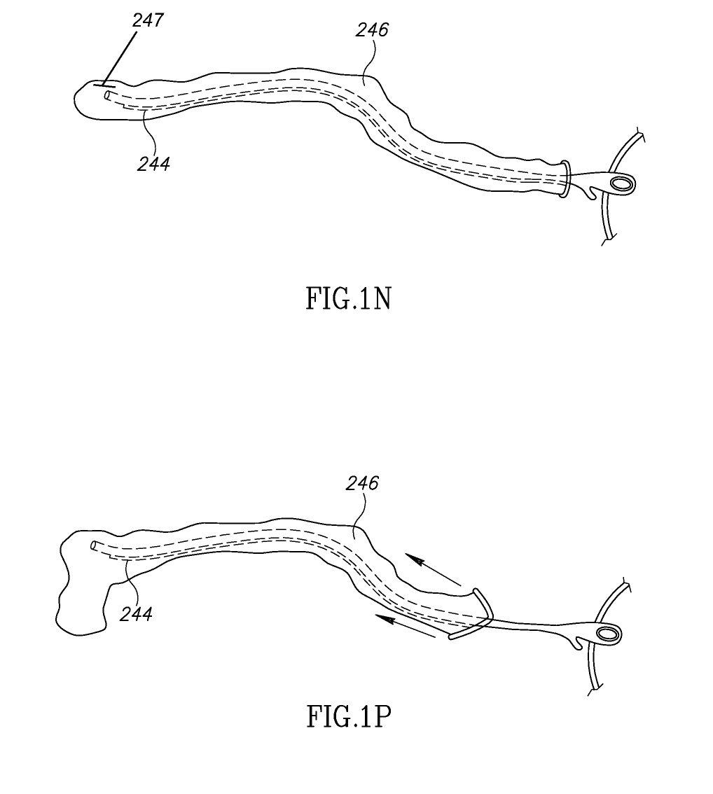

Reference is now made to FIGS. 1A-1D, which are simplified illustrations of an endoscopic device 100, constructed and operative in accordance with an embodiment of the present invention. The endoscopic device 100 is shown here as an add-on accessory that assembles with an endoscope (such as but not limited to, a colonoscope), such as endoscope 212 shown in FIGS. 1G-1I.

Referring to FIGS. 1A-1B, endoscopic device 100 (not shown in FIGS. 1A-1B) includes a washing member 201, which may include a distal ring 202 with a fastening device 205, such as fastening ears 205, for snugly fitting on the distal end of an endoscope 212 (FIGS. 1G-1I). The ring 202 has an inlet port 203 which fluidly communicates with outlet ports 204 distributed on the distal face of ring 202. As will be explained below, a cleaning jet (or washing flow) of fluid flows into inlet port 203 and out outlet ports 204.

Referring to FIGS. 1C-1D, endoscopic device 100 includes a suction member 206, preferably shaped to conform over the outer cylindrical contour of the endoscope. Suction member 206 includes a cleaning jet lumen 207, a drain lumen 208 (also referred to as suction lumen 208), and a debris clearing lumen 209 that fluidly communicates with the side of drain lumen 208 for providing a jet spray to clear debris from drain lumen 208. Cleaning jet lumen 207 is fluidly connected to inlet port 203 of washing member 201 (FIGS. 1A-1B) with a tube 210 as seen in FIGS. 1G-1I.

The jet spray used to clear the debris is just one example of an anti-clogging element that moves with respect to the washing member 201 and/or suction member 206 for clearing an obstruction from the washing member 201 and/or suction member 206. Alternatively, instead of a fluid element (fluid meaning liquid or gas), the anti-clogging element could be a solid element, such as a probe, needle, pin or other mechanical element, that moves with respect to the washing member 201 and/or suction member 206 for clearing an obstruction from the washing member 201 and/or suction member 206.

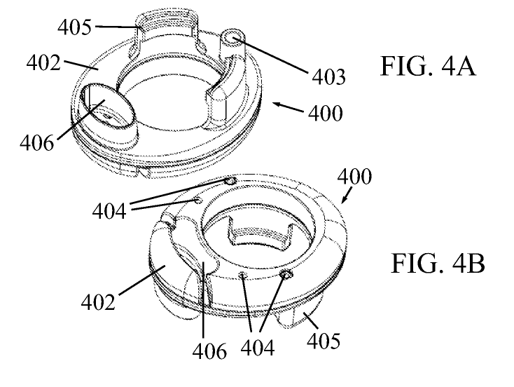

Reference is now made to FIGS. 4A-4B, which illustrate an endoscopic device 400, constructed and operative in accordance with another embodiment of the present invention. Endoscopic device 400 includes a combination washing and suction member, which may include a ring 402 with a fastening device 405, such as fastening ears 405, for snugly fitting on the distal end of endoscope 212 (not shown here). The ring 402 has a washing inlet port 403 for the cleaning jet (or washing flow) of fluid to flow through to one or more outlet ports 404 positioned on the ring 402. The outlet ports 404 may be of different sizes and shapes with varying jet flow strength and spreading, and may direct the jets/flows in different directions, thus allowing the breakdown of waste (that may be of solid nature) and a thorough washing and cleaning of the body lumen walls, by using minimal jet pressure and avoiding or minimizing any damage to the body lumen. Jet/flow can be continuous or non continuous, or in pulses. All washing jets/flows parameters may be controlled manually or automatically via the controller. A suction port 406 is provided for sucking and draining debris, body liquids and washing liquids. This embodiment operates in a similar fashion as the previous embodiment, with the washing and suction members combined on one piece.

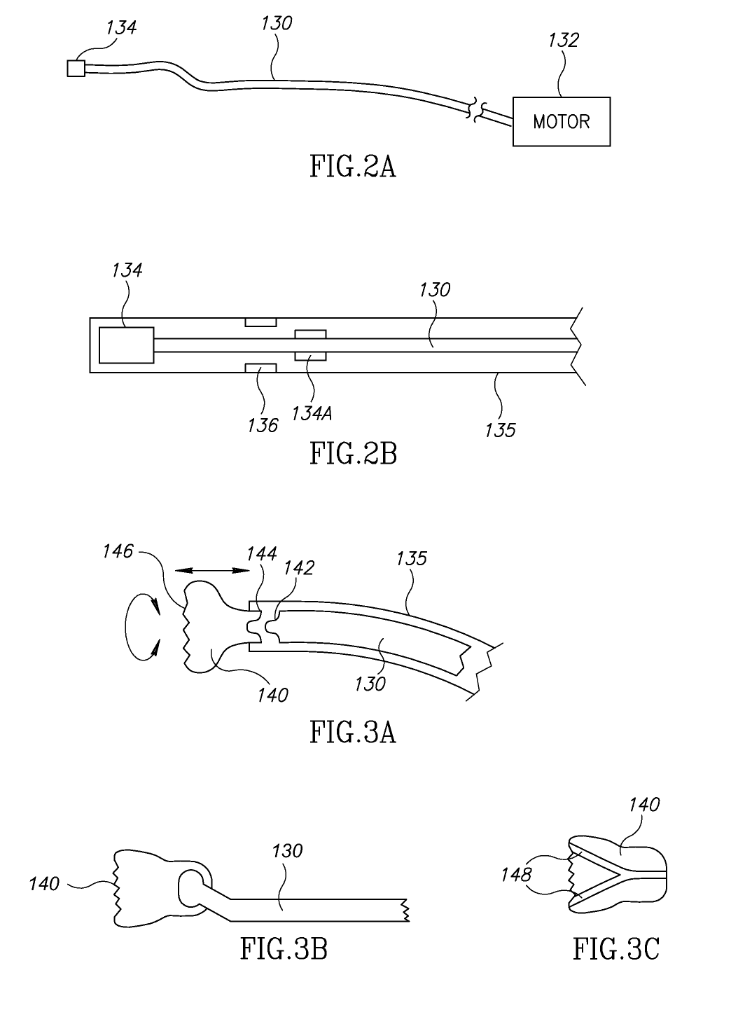

Referring to FIGS. 1E-1F, endoscopic device 100 includes a connector head 211, configured to be mounted on the proximal end of endoscope 212. Connector head 211 includes a cleaning jet port 213, a drain lumen port 214, and a debris clearing lumen port 215 that fluidly communicate respectively with cleaning jet lumen 207, drain lumen 208, and debris clearing lumen 209 of suction member 206 with tubing 216 (FIG. 1J). Connector head 211 may have a housing 217 made of two parts, wherein one of the parts has the ports 213-215 and the other part has corresponding inlet ports 218-220, respectively in fluid communication with ports 213-215. Connector head 211 may have additional ports for additional functions, such as but not limited to, working channels (large and/or small), additional irrigation and/or suction lumens and controls, additional features as described in FIGS. 1K-1M and in FIGS. 2A-3C.

Reference is now made to FIGS. 1G-1I, which illustrate different ways of securing endoscopic device 100 on endoscope 212. In FIG. 1G, endoscopic device 100 is secured to endoscope 212 with one or more closed or full rings 221, such as at an interval of about 20 cm. The closed rings 221 are preferably smooth and rounded to easily glide through the GI tract.

In FIG. 1H, endoscopic device 100 is secured to endoscope 212 with one or more open or partial rings 222 (the rings may span, for example, about 300°). The rings may be attached by snapping on to the endoscope.

In FIG. 1I, endoscopic device 100 is secured to endoscope 212 with one or more adhesive tape strips 223. The adhesive tape strips 223 are used to tape down a tube 228 that carries the fluid tube connections between connector head 211 and suction member 206.

The sleeve mentioned further below is an alternative means for securing the device to the endoscope.

Reference is now made to FIG. 1J, which illustrates a control system 230 for operating endoscopic device 100, constructed and operative in accordance with another embodiment of the present invention.

Control system 230 may be housed in a control cabinet and may include, without limitation, a power supply 231, controller (processor) 232, one or more pumps 233 (e.g., peristaltic pumps) and one or more control valves 234 (e.g., a pinch valve). Valve 234 is connected to a suction source and controls the suction to the drain lumen 208 of suction member 206. The pumps 233 are used to pump fluid in or out of connector head 211 and suction member 206 and to pump cleaning fluid to washing member 201. The pumps and valve are connected to connector head 211 with tubing 235. The one or more valves 234 may allow switching pressurized washing liquid into the suction drain lumen for unclogging any clog. Such action can be controlled manually or automatically once the controller senses a clog, or in a sequential automatic preventive manner.

The operation of endoscopic device 100 includes pumping a cleaning jet of fluid to washing member 201 for cleaning the GI tract during passage therethrough, typically at low pressure, such as but not limited to, about 2 bar. Waste material (which may flow together with the washing liquid) is sucked into drain lumen 208 and exits to a waste receptacle (not shown) which is preferably disposable.

The cleaning jet can be directed in a variety of directions, such as but not limited to, distally (i.e., forward) to break up clumps and solid waste, proximally (i.e., backwards towards the anus) and to the sides, at angles, or any combination thereof. Control system 230 may be used (e.g., by means of a fluid manifold, fluid switch, electronic valve, etc.) to selectively direct the fluid flow to the cleaning jet lumen 207, drain lumen 208, or debris clearing lumen 209, and/or to selectively direct the jet flow to the forward, side or rear openings of the device. The jet spray can be used to break up and clear away debris from the drain outlet to ensure proper drainage of the fluid used to clean the GI tract. The size of the suction inlet is preferably somewhat smaller than the rest of the path through to the suction outlet so that once debris has passed the inlet it should not cause a clog with the suction lumen.

The jet fluid ensures that any debris, which has become stuck at the suction inlet so as to clog the inlet (because of size or shape), is cleared away from blocking the suction inlet by the force and direction of the flow. The jet fluid avoids sucking the body lumen wall into the suction inlet thus avoiding clogging and potential damage to the body lumen, and also preventing the sucked wall from arresting movement of the endoscope.

A variety of sequence of operations can be used in employing the endoscopic device 100, such as but not limited to, different sequences of alternating between spraying jets of fluid to clean away debris in the GI tract, sucking the debris away, cleaning debris away from the drain outlet, including different time durations of each part of the sequence, frequency and amplitude of the jet sprays and suction, all of which may be automatically controlled by the controller or manually controlled by the operator.

In another embodiment of the invention, all the above-mentioned features/channels/lumens/cleaning jets/suction and the like of the endoscopic device are incorporated within the endoscope itself, that is, supplied or built together with the endoscope by the endoscope manufacturer, or inserted through one or more of available channels within an endoscope.

The lumen for the jet flow to clean the drain may be incorporated in a working channel for passing therethrough working tools (for cutting tissue, collecting biopsy material, etc.).

The work flow may be continuous with a jet flow and suction. Alternatively, the work flow may alternate between jet flow and suction. The work flow may comprise pulses of jet flow, synchronized or not.

The jet flow may be directed proximally to create jet propulsion to propel, or assist in propelling, the endoscope distally into the GI tract. The jet flow may be continuous with suction (and may be used concurrently with the jet flow for cleaning). Alternatively, the work flow may alternate between jet flow and suction. The work flow may comprise pulses of jet flow, synchronized or not.

The suction may take place at a distal portion of the device, on the sides (via suction openings) and/or at a proximal portion of the device, or a combination thereof.

Reference is now made to FIGS. 1K-1M. Grinding or chopping members may be provided (preferably, but not necessarily, in the drain lumen, not shown in FIGS. 1K-1M) to assist in reducing the size of the debris for draining/flushing away, such as but not limited to, a grinding feed screw 236 (FIG. 1K) of the kind used in food or meat grinders that extends partially or fully the length of the drain lumen (there may be further side openings 238 seen in FIG. 1L to allow debris to enter through the sides to be ground by the grinding screw 236), chopper blades 240 (FIG. 1M) situated at the opening to the drain lumen, or vibrating transducer or ultrasonic transducer 242 (shown in dotted lines in FIG. 1L) situated at the opening to the drain lumen or at the head of the device or at the head of the endoscopic device 100 for liquefying/dissolving solid waste together with, or without, washing liquids with, or without, the mechanical assistance of cleaning jets, breaking up debris and for mechanical assistance of draining the debris and fluids out of the body lumen. The mechanical draining may be combined together with suction or may be performed without suction.

Jet steering, jet propulsion or percussion propulsion may be used with the endoscopic accessory.

Reference is now made to FIGS. 1N and 1P. The endoscopic accessory maintains sterility by using a sheath 244 that fits over the endoscope (the sheath is also referred to as a sleeve). The fluid lumens of the endoscopic accessory and working channels can be external to the sheath and not come into contact with the endoscope. The sheath is discarded after use.

Since a new sheath is placed over the endoscope each time, even if the sheath has a puncture or tear and the endoscope is contaminated, the following patient is protected by a new sheath, thus providing a double safety protection to any treated patient.

As an added protection, as part of the cleaning process of the device after treatment is finished, a second sheath 246 may be placed over the first sheath 244. The second sheath 246 covers and seals the first sheath 244 so that any potential contamination on the first sheath 244 is sealed within the second sheath 246. The far end of the second sheath 246 is attached and sealed onto the far end of the first sheath 244, thus trapping any potential contamination within the closed and sealed sheaths. (The sheaths can be removed from the endoscope by inverting them backwards over the endoscope head, thereby preventing the endoscope from contacting any contaminants.) In this way contamination of the endoscope is prevented. Contamination of the surroundings is also prevented when removing the sheaths. The disposable sheaths once disposed are also sealed thus causing no danger of contamination to any person and equipment involved after completing the colonoscopic procedure.

In an alternative embodiment, the channels are inside the sleeve, and may even be attached to, or part of the sleeve. The sterility of the endoscope is maintained by aseptically sealing the assembly. The sheath may have a transparent window 247 (FIG. 1N) to allow illumination, imaging and other functionalities using other technologies and devices (e.g., ultrasonic, radio, laser, and others), while the endoscope remains fully covered, isolated and sterile.

The disposable sleeve may contain also one or more working channels to allow medical procedures while keeping the endoscope sterile. The additional working channels may be sufficiently wide for insertion of large tools.

In yet another alternative embodiment, the sleeve itself may be fully transparent allowing undisturbed imaging by the endoscope. In such case, if a working channel of the endoscope is to be used by a surgical tool, or any other tool, the sleeve may be pierced and penetrated by the tool allowing the necessary procedure. Such piercing is doable also during the procedure while the devise is inside the body lumen.

Reference is now made to FIG. 2A, which illustrates a vibratory device 130, which may be used with endoscopic device 100. As will be described below, vibratory device 130 can set up vibrations in endoscopic device 100 to help advance endoscopic device 100 in the GI tract. In addition, the vibrations can help loosen and remove debris from the body lumen and from its side walls. Any combination and sequence of irrigation/washing, vibration and suction can be used to enhance cleaning the colon (or any body lumen) even if minimal preparation or no preparation was done. The induced vibration may also assist the linear forward/backward movement of the endoscope. The vibration helps release the endoscope from the surrounding body lumen, and thus helps move the endoscope through the body lumen during the procedure. Control of the process/procedure can be done manually, or automatically by the system controller. The mode of operation can be discrete on/off or with a full range of proportional control.

The frequency and magnitude of the vibrations can be controlled for the particular situation. Alternatively or additionally, vibratory device 130 can be used in a percussion mode. The motor can be situated within the cabinet unit via proper mechanical connector, or even on or within the endoscope and even at the tip of the endoscope. The motor may be a miniature motor to allow attaching to, or contained within, the endoscope or endoscope tip.

In the illustrated embodiment, vibratory device 130 includes a slender wire that extends partially or fully the length of the cleaning jet lumen, drain lumen, or debris clearing lumen, or which is external to and separate from these lumens (and also can be put inside any working channel of the endoscope, or incorporated into the structure of an endoscope, becoming part of the endoscope). A motor 132 maybe connected to vibratory device 130 for rotation thereof. Vibratory device 130 may be formed with one or more off-center kinks or curves, such as but not limited to, S-shaped curves, such that during rotation the slender wire causes oscillation or any other kind of vibration of endoscopic device 100, along all or part of its length. The wire may be constructed of a material which is sufficiently rigid to create the vibrations, but flexible enough to navigate the various turns of the GI tract (and not disturb/degrade the flexibility and controllability of the endoscope by the user). Examples of suitable materials include, but are not limited to, stainless steel (AISI 316) and nitinol.

The motor may vibrate the vibratory device 130 by rotation motion, linear tangential motion, linear longitudinal motion, or any combination thereof.

Alternatively or additionally, motor 132 may be a percussion type of motor (like a percussion drill motor, for example). When the motor 132 is activated it causes a piston 134 at the distal end of vibratory device 130 to strike a distal end of the endoscopic device 100, thereby causing the endoscope, with endoscopic device 100 attached thereto, to advance distally in the body lumen, similarly to that described above with reference to FIG. 2A.

Referring to FIG. 2B, one or more pistons 134A may be disposed along the length of vibratory device 130 and are arranged to strike stops 136 affixed at different stations along a lumen or guide member 135 in which vibratory device 130 is disposed. The system is propelled by multiple local striking forces distributed along the lumen 135. The distribution of multi-propulsion forces along the length of the guide member 135 enables propelling very long and twisted guide members (differently shaped and sized) along and through very long and twisted_body lumens and passages. The striking forces can be controlled for forward motion or backward motion by the pistons 134A striking stops 136 in the forward or backward direction.

It is noted that the vibratory device, pistons and other elements of FIGS. 2A-2B may be part of the cleaning device (disposable), or part of an endoscope/colonoscope in general. They can be assembled in, or inserted into, one of the described channels (irrigation, suction, etc.) or in a separate additional channel, or even within an existing channel of an endoscope, such as but not limited, a working channel.

Vibratory device 130 may be constructed with screw threads and/or grinding or chopping members, similar to the embodiment of FIGS. 1K-1M to assist in reducing the size of the debris for draining/flushing away and for avoiding clogging. The operation of motor 132 may be controlled by a controller (such as controller 232 above) that can change the rotational direction of vibratory device 130, as well as the frequency and magnitude of the vibrations. For example, while turning in one given direction, e.g., counterclockwise, vibratory device 130 may move debris proximally towards the anus for expelling the debris from the body. However, vibratory device 130 may also intermittently turn in the opposite direction, which may help loosen debris and clear out blockages before continuing to rotate in the direction for drawing debris out of the body. The controller may sense with appropriate sensors, such as vacuum sensors, flow sensors, torque sensors, sensors that sense slow down of spin, and the like (not shown), the presence of such blockages to decide whether to change the rotational direction.

Of course, sensing clogging or blocking can be done even if vibratory devise 130 is not implemented in the system; rather the sensors may be used in simple configurations just with irrigation and suction. Once clogging is sensed, the system may automatically anti-clog the device with a variety of means, such as but not limited to, changing the irrigation/suction routine/sequence/frequency, or increasing suction and/or irrigation pressures and/or flows, or reversing pressure and flow in the opposite directions to release blockages, or more aggressive means, such as vibration, percussion, or changing rotation direction and magnitude.

In accordance with an embodiment of the invention, a similar method of clogging removal may be carried out (as described above with reference to FIGS. 1C-1J) wherein the suction is periodically replaced with an opposite/positive pressure that pushes back any clogging debris. This can be implemented once clogging is sensed or alternatively in a periodically fashion so that no sensor may be needed.

In this and other embodiments of the invention, lubricants, dyes, marking substances, and/or drugs or other treatment substances may be introduced into guide member 135 and sprayed into the GI tract, e.g., through the cleaning jet lumen or any other port dedicated or not dedicated for such a purpose. Alternatively or additionally, surgical tools, e.g., for cutting and collecting polyps or other tissues for biopsies, may be introduced via guide member 135 or any additional channels. External attachment to the endoscope allows adding more and larger channels and working channels for any necessity.

Various accessories may be mounted at the distal end of vibratory device 130 to enhance its cleaning capability (e.g., to break up debris) and propulsion capability. Some examples are now described with reference to FIGS. 3A-3C.

FIG. 3A illustrates that the distal end of vibratory device 130 may be configured to mate with a grinding tool head 140. For example, the distal end of vibratory device 130 may be configured with a male coupling 142 which mates with a corresponding female coupling 144 at the proximal end of grinding tool head 140. Pushing the distal end of vibratory device 130 towards grinding tool head 140 causes male coupling 142 to mate with female coupling 144 so that the rotation and/or percussive motion of vibratory device 130 is imparted to grinding tool head 140. Of course other couplings may be employed. It is noted that the user can decide when to couple vibratory device 130 to grinding tool head 140 (they do not have to be coupled all the time, although they can be).

The grinding tool head 140 of FIG. 3A may comprise a plurality of grinding teeth, serrations, protrusions, sharp points, abrasive elements and the like, generally designated by the reference numeral 146 in FIG. 3A.

FIG. 3B illustrates a variation of the embodiment of FIG. 3A. In this embodiment, the distal end of vibratory device 130 is bent off-center and couples with grinding tool head 140. In the illustrated embodiment the coupling is different from that of FIG. 3A, but as mentioned before, any coupling may be used. The off-center configuration of the distal end of vibratory device 130 enhances the oscillatory motion of grinding tool head 140.

FIG. 3C illustrates a variation of grinding tool head 140 for use when vibratory device 130 extends partially or fully through the cleaning jet lumen. In this embodiment, grinding tool head 140 is formed with channels 148 for cleaning fluid to flow therethrough. Channels 148 are in fluid communication with the cleaning jet lumen so that the cleaning jet of fluid (e.g., pressurized water) that flows into the cleaning jet lumen flows out through channels 148. As described before, the cleaning jet can be directed in a variety of directions to break up clumps and solid waste, for example.

Instead of the invention being packaged as an add-on accessory, the invention may be built into the endoscope and be provided as part of the endoscope by the endoscope manufacturer.

The invention obviates the need for the aggravating pre-cleaning procedures widely used for preparing for a colonoscopy—no need for enemas, drinking awful tasting glop, diarrhea, etc. Rather the invention starts cleaning the colon upon entering the anus without causing discomfort to the patient. Imaging may be performed while passing the instrument into the colon or while extracting the instrument from the colon.

The following is a summary of a typical, but non-limiting, procedure of using the endoscopic device 100. First, the patient may either skip any pre-cleaning of the colon altogether or may undergo a much shorter and friendlier pre-cleaning, such as a small dosage of an enema.

A sterilized endoscope is prepared by putting/attaching a sterile endoscopic device 100 onto the endoscope (in the case of an add-on accessory). Alternatively, the endoscopic device 100 is pre-manufactured with the endoscope as a built-in manufactured assembly, in which case the manufactured assembly comes sterilized or is sterilized prior to use. Sheath 244 can be part of endoscopic device 100 to maintain sterility.

The endoscope covered with endoscopic device 100 is connected to the endoscope cabinet and the connector head 211 of endoscopic device 100 is connected to the control system 230. Alternatively the endoscope with the endoscopic device 100 built-in is connected to the control system 230. The system is now ready to start the medical procedure.

Next, the endoscope with the endoscopic device 100 is inserted in the colon. Fluid is introduced in the endoscopic device 100 and the jet of fluid starts to clean the path for the endoscope in the colon. The endoscope may take images of the colon as the endoscope enters the colon and/or as the endoscope exits the colon. Any waste material may be collected in a waste container. After imaging and/or other procedures performed with tools through the working channel, the endoscope with endoscopic device 100 is removed from the colon. The second sheath 246 is placed over the first sheath 244 so that any potential contamination on the first sheath 244 is sealed within the second sheath 246. The sheaths are removed from the endoscope by inverting them backwards over the endoscope head, thereby preventing the endoscope and surroundings from contacting any contaminants. The disposable sheaths are then disposed. Any waste material collected during the colonoscopy is sealed in the waste container and disposed.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

What is claimed is:

1. A method for enhancing performance of an endoscope (212) for use with a body lumen that has not been sufficiently cleaned for viewing with said endoscope (212), comprising:
assembling an endoscopic device (100, 400) to an endoscope (212), said endoscopic device (100, 400) comprising a washing member (201, 404) that provides a flow of cleaning fluid for cleaning a body lumen, and a suction member (206, 406) for draining material from the body lumen; and
causing fluid to exit said washing member (201, 404) into the body lumen to clear debris away from a path of said endoscope (212) so that the endoscope (212) can view the exposed body lumen, and draining at least a portion of the debris through said suction member (206, 406).

2. The method according to claim 1, wherein assembling the endoscopic device (100, 400) to the endoscope (212) comprises assembling the endoscopic device (100, 400) as an add-on accessory to the endoscope (212).

3. The method according to claim 1, wherein assembling the endoscopic device (100, 400) to the endoscope (212) comprises assembling the endoscopic device (100, 400) with the endoscope (212) as a built-in manufactured assembly.

4. The method according to claim 1, further comprising clearing an obstruction from at least one of said washing member (201, 404) and said suction member (206, 406).

5. The method according to claim 4, comprising causing fluid to exit at least one of said washing member (404) and said suction member (206) to clear the obstruction.

6. The method according to claim 1, further comprising causing fluid to exit said washing member (201, 404) to help propel said endoscope (212) in the body lumen.

7. The method according to claim 1, further comprising introducing vibratory motion in said endoscope (212) to help propel said endoscope (212) in the body lumen.

8. The method according to claim 1, further comprising introducing percussion forces in said endoscope (212) to help propel said endoscope (212) in the body lumen.

9. The method according to claim 1, further comprising fitting a discardable sheath (244, 246) over the endoscope (212).

10. The method according to claim 9, further comprising removing said sheath (244, 246) from the endoscope (212) by inverting said sheath (244, 246) backwards over an end of the endoscope (212).

11. The method according to claim 1, further comprising performing a sequence of alternating between spraying jets of fluid through said washing member (201, 404) to clean away debris in the body lumen, sucking the debris away through said suction member (206, 406), and cleaning debris away from a drain outlet of said suction member (206, 406).

12. The method according to claim 11, further comprising using a control system (230) to automatically control operational parameters of the sequence, the operational parameters comprising at least one of time durations of each part of the sequence, and frequency and amplitude of spraying the jets of fluid and sucking the debris.

13. An endoscopic device (100, 400) for use with an endoscope (212), comprising:
a washing member (201, 404) that provides a flow of cleaning fluid for cleaning a body lumen;
a suction member (206, 406) for draining material from the body lumen; and
an anti-clogging element that moves with respect to at least one of said washing member (201, 404) and said suction member (206, 406) for clearing an obstruction from at least one of said washing member (201, 404) and said suction member (206, 406).

14. The endoscopic device (100, 400) according to claim 13, wherein said anti-clogging element comprises a spray of fluid that exits said washing member (404) or said suction member (206).

15. The endoscopic device (100, 400) according to claim 13, further comprising a control system (230) for operating said endoscopic device (100, 400), said control system (230) comprising a controller (232), one or more pumps (233) and a control valve (234), wherein said control valve (234) controls suction to said suction member (206) and said one or more pumps (233) pump cleaning fluid to said washing member (201).

16. The endoscopic device (100, 400) according to claim 13, further comprising a discardable sheath (244, 246) that fits over the endoscope (212).
